(19) United States
(12) Patent Application Publication
(10) Pub. No.: US 2021/0038184 A1
(43) Pub. Date: Feb. 11, 2021

(54) ULTRASOUND DIAGNOSTIC DEVICE AND ULTRASOUND IMAGE PROCESSING METHOD

(71) Applicant: Hitachi, Ltd., Tokyo (JP)

(72) Inventor: Atsushi Shiromaru, Tokyo (JP)

(73) Assignee: Hitachi, Ltd., Tokyo (JP)

(21) Appl. No.: 16/896,547

(22) Filed: Jun. 9, 2020

(30) Foreign Application Priority Data
Aug. 8, 2019 (JP) 2019-146107

(51) Int. Cl. A61B 8/08; A61B 8/14; A61B 8/00

(52) U.S. Cl. CPC A61B 8/0833; A61B 8/14; A61B 8/469; A61B 8/5238; A61B 8/463

(57) ABSTRACT

A region of interest extending in the depth direction is set in the center portion of a tomographic image. Identification processing is applied on an image portion defined by the region of interest, on a frame-by-frame basis. In the identification processing, pattern matching processing is performed at positions within the region of interest using a template. Based on a plurality of correlation values obtained from this processing, a tissue image that satisfies identification conditions is identified. In the pattern matching processing, a set of templates may be used.

### CROSS REFERENCE TO RELATED APPLICATION

This application claims priority to Japanese Patent Application No. 2019-146107 filed on Aug. 8, 2019, which is incorporated herein by reference in its entirety including the specification, claims, drawings, and abstract.

### TECHNICAL FIELD

The present disclosure relates to an ultrasound diagnostic device and an ultrasound image processing method, and in particular to a technique of identifying a particular tissue image included in an ultrasound image.

### BACKGROUND

An ultrasound image diagnostic device is a medical device that forms an ultrasonic image based on received signals obtained by transmitting and receiving ultrasound waves to and from a living body. The ultrasound diagnostic device has an ultrasound probe, and a probe head of the ultrasound probe transmits and receives ultrasound waves. Specifically, while an examiner holds the probe head and causes a wave transmitting and receiving surface of the probe head to abut against the surface of the living body, an ultrasound transducer in the probe head transmits and receives ultrasound waves. When a position and a posture of the probe head are changed, content of an ultrasound image change accordingly. In that case, for example, in the ultrasound image, a position of a tissue image changes, or a tissue image that has been visible until then disappears, and another tissue image appears.

### SUMMARY

### Technical Problem

If, in order to identify a particular tissue image included in an ultrasound image (hereinafter, referred to as a “target tissue image”) automatically, a search for the target tissue image is conducted in the entire ultrasound image, another, similar tissue image is likely to be mistakenly identified as the target tissue image. It is thus desirable to reduce the possibility of occurrence of such erroneous identification. It is also desirable to enable easy cancellation of the identified state by the user when such erroneous identification has been made.

Patent Document 1 (JP 2017-104248 A) discloses an ultrasound diagnostic device that automatically performs a series of processing steps including automatic recognition of a measured surface. Patent Document 2 (JP 2018-149055 A) discloses a technique of pattern matching. Neither patent document discloses a technique for enhancing the accuracy in identifying a target tissue image when the target tissue image and other similar tissue images are mixed.

An object of the present disclosure is to enhance the accuracy in identifying a target tissue image. Alternatively, an object of the present disclosure is to enable, when another tissue image which is not the target tissue image has been identified, easy cancellation of the tissue image.

### Solution to Problem

An ultrasound diagnostic device according to the present disclosure includes a probe head that transmits and receives ultrasound waves, an image forming unit that forms an ultrasound image based on a received signal output from the probe head, a region setting unit that defines a region of interest extending in the depth direction with respect to the ultrasound image, an identification unit that identifies, in an image portion defined by the region of interest, a tissue image that satisfies identification conditions, and a tissue marker generation unit that generates, when the tissue image satisfying the identification conditions is identified, a tissue marker indicating the tissue image and causes the tissue marker to be displayed on the ultrasound image, and in this device, when the tissue image that has been identified so far is outside of the image portion in accordance with operation of the probe head, the tissue image is excluded from identification targets.

An ultrasound image processing method according to the present disclosure includes the steps of setting, with respect to an ultrasound image, a region of interest extending on a center line of the ultrasound image in the depth direction, the ultrasound image being formed based on a received signal output from a probe head transmitting and receiving ultrasound waves; identifying, in an image portion defined by the region of interest, a tissue image that satisfies identification conditions; displaying a region marker indicating the region of interest on the ultrasound image; and displaying a tissue marker indicating, on the ultrasound image, an identified state of the tissue image satisfying the identification conditions.

### BRIEF DESCRIPTION OF DRAWINGS

An embodiment of the present disclosure will be described based on the following figures, wherein:

FIG. 1 is a block diagram showing a configuration example of an ultrasound diagnostic device according to an embodiment;

FIG. 2 is a block diagram showing a configuration example of an identification unit;

FIG. 3 shows pattern matching processing;

FIG. 4 shows an identified target tissue image;

FIG. 5 shows a mistakenly identified tissue image;

FIG. 6 shows cancellation of an identified state by operation of a probe head;

FIG. 7 shows a result of extraction processing of a tissue image included in volume data;

FIG. 8 shows measurement after identification processing;

FIG. 9 shows a set of templates;

FIG. 10 is a flowchart showing the identification processing;

FIG. 11 is a flowchart showing an example of subsequent processing;

FIG. 12 is a flowchart showing another example of the subsequent processing;

FIG. 13 shows a second example of a region of interest;

FIG. 14 shows a third example of the region of interest; and

FIG. 15 shows a fourth example of the region of interest.

### DESCRIPTION OF EMBODIMENTS

Hereinafter, an embodiment will be described with reference to the drawings.

### (1) Summary of Embodiment

An ultrasound diagnostic device according to the embodiment includes a probe head, an image forming unit, a region setting unit, an identification unit, and a tissue marker generation unit. The probe head transmits and receives ultrasound waves. The image forming unit forms an ultrasound image based on a received signal output from the probe head. The region setting unit defines a region of interest extending in the depth direction with respect to the ultrasound image. The identification unit identifies, in an image portion defined by the region of interest, a tissue image that satisfies identification conditions. When the tissue image satisfying the identification conditions is identified, the tissue marker generation unit generates a tissue marker indicating the tissue image which is in the identified state.

According to the above configuration, if a tissue image satisfying the target conditions is included in the image portion of the ultrasound image, that tissue image is identified automatically. Such an identified state can be easily achieved by adjusting a position and a posture of the probe head. At this time, no extra burden is imposed on the examiner. The examiner can recognize the identified state and the identified tissue image through observation of the tissue marker. If the identified tissue image is erroneous; that is, if the identified tissue image is not the target tissue image, the position and the posture of the probe head only need to be changed so that that tissue image can be outside of the image portion. Thus, the tissue image is naturally excluded from identification targets. No special input operation, such as button operation, is necessary to change the identification targets. As such, with the above configuration, it is possible to select an identification target easily by operating the probe head.

It is easy to translate and rotate a scanning plane while maintaining the orientation of the scanning plane by operating the probe head. On the other hand, it is impossible to move the entire scanning plane to the deeper side or the shallower side by operating the probe head. The shape of the region of interest, and thus, the shape of the image portion, are determined in consideration of such conditions specific to ultrasound diagnosis.

In the embodiment, the region of interest functions as a basis in searching for a tissue image satisfying the identification conditions. The image portion described above is a portion that is actually referred to when a search is conducted based on the region of interest. The image portion is, for example, a region that is of a size larger than the region of interest, or an internal region of the region of interest. When the lateral width of the region of interest is increased, the possibility that tissue images other than the target tissue image enter the image portion increases. On the other hand, when the lateral width of the region of interest is reduced, the target tissue image tends to be outside of the image portion, or operation of enclosing the target tissue image within the image portion becomes difficult. Therefore, it is desirable to keep the lateral width of the region of interest appropriate.

In the embodiment, the region of interest is provided on the center line of the ultrasound image and has an elongated shape extending along the center line. Upon observation and measurement of the target tissue image, the position and the posture of the probe head are usually adjusted so that the target tissue image is positioned at the center portion along the right and left directions in the ultrasound image. On the other hand, although the depth at which the target tissue image is positioned is a generally in a center portion along the depth direction, the target tissue image may be positioned at a slightly shallower position or at a slightly deeper position. The above configuration takes these assumptions into consideration. Specifically, in the embodiment, the ultrasound image has a fan shape, and the region of interest has a rectangular shape separated away from the upper edge and the lower edge of the ultrasound image.

The identification conditions are conditions for recognizing a tissue image as a target tissue image. For example, one tissue image that has been evaluated as the best image is determined to be a target tissue image. A plurality of tissue images may also be determined to be target tissue images satisfying the identification conditions.

In the embodiment, the identification unit performs identification processing on a frame-by-frame basis. In the identification processing on a frame-by-frame basis, pattern matching processing is performed at positions within the region of interest using at least one template, and a tissue image satisfying the identification conditions is identified based on a plurality of pattern matching results obtained from this pattern matching processing.

In the embodiment, the pattern matching processing uses a set of templates that includes a plurality of templates different from one another. The plurality of types of templates corresponding to various appearances of target tissue images are prepared so that the target tissue image can be recognized regardless of the appearance it may present. For example, if the target tissue image is a blood vessel image, it is desirable to prepare a plurality of templates corresponding to a cross section, a vertical section, a diagonal section, etc. of the blood vessel image.

In the embodiment, the set of templates includes a template that simulates a tissue image with a shadow. Generally, when viewed from the probe head side, echoes coming from behind (the backside of) a massive tissue are weak, and such a tissue tends to have a shadow behind it. The above configuration prepares the template in consideration of such a shadow.

In the embodiment, the pattern matching processing at the positions within the region of interest includes at least one of change in template size, change in template rotation angle, and template deformation. The set of templates may include a template which does not require rotation. The concept of template deformation includes changing the ratio between the vertical size and the lateral size.

The ultrasound diagnostic device according to the embodiment includes a region marker generation unit that generates a region marker indicating the region of interest and then causes the region marker to be displayed on an ultrasound image. According to this configuration, it becomes easier to recognize the region of interest and the image portion defined by the region of interest in contrast to the entire ultrasound image. The image portion is a portion that corresponds to the region of interest or that can be identified as the region of interest, and therefore, the region marker is a marker that also indicates the image portion or a rough position of the image portion.

An ultrasound image processing method according to the embodiment includes a first step, a second step, a third step, and a fourth step. In the first step, with respect to an ultrasound image formed based on a received signal output from a probe head transmitting and receiving ultrasound waves, a region of interest extending on the center line of the ultrasound image in the depth direction is set. In the second step, a tissue image that satisfies identification conditions is identified in an image portion defined by the region of interest. In the third step, a region marker indicating the region of interest is displayed on the ultrasound image. In the fourth step, a tissue marker indicating the identified state of the tissue image satisfying the identification conditions is displayed on the ultrasound image.

The above ultrasound image processing method can be realized as hardware functions and software functions. In the case of the latter, a program for executing the ultrasound image processing method is installed in an information processing device via a non-transitory storage medium or a network. The concept of the information processing device encompasses an ultrasound diagnostic device, an ultrasound image processing device, a computer, and the like.

### (2) Details of Embodiment

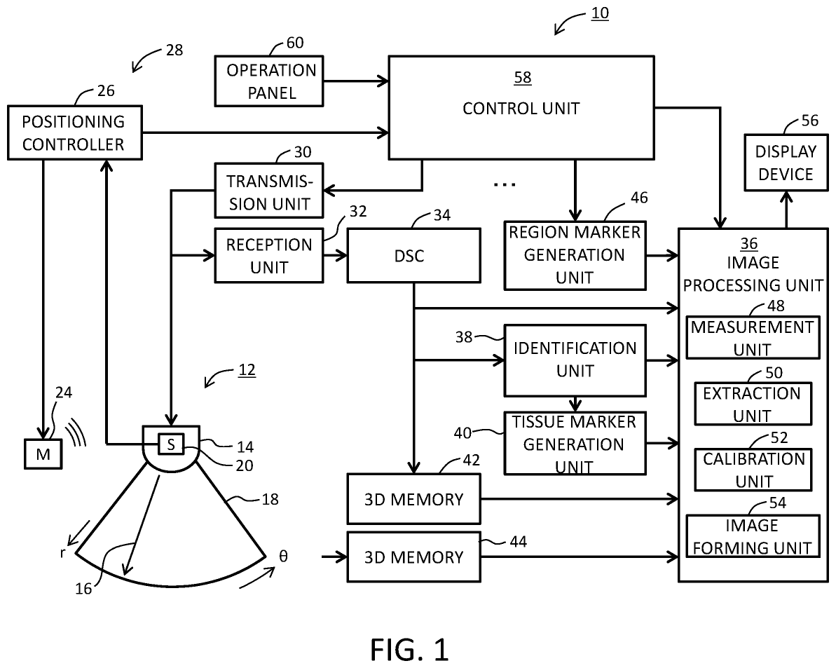

As shown in FIG. 1, an ultrasound image diagnostic device is a medical device that is provided in a medical institution, such as a hospital, and forms an ultrasound image by transmitting and receiving ultrasound waves to and from a subject as a living body. The ultrasound diagnostic device is roughly composed of a device body 10 and an ultrasound probe 12. The ultrasound probe 12 is detachably connected to the device body 10.

The ultrasound probe 12 is composed of a probe head 14, a cable, and a connector. The cable and the connector are omitted in the drawing. The probe head 14 is a portable transducer. The probe head 14 is held by an examiner who is a user. An array of transducer elements is provided in the probe head 14. Specifically, the array of transducer elements is a one-dimensional array of transducer elements which is a plurality of transducer elements arranged in an arcuate shape. The array of transducer elements transmits and receives ultrasound waves, thereby forming an ultrasonic beam 16.

A scanning plane 18 is formed by electronic scanning of the ultrasound beam 16. In FIG. 1, r indicates the depth direction and θ indicates an electronic scanning direction. As electronic scanning methods, an electronic linear scanning method, an electronic sector scanning method, and the like are known. In the embodiment, an electronic convex scanning method which is one aspect of the electronic linear scanning method is adopted. An array of transducer elements that includes a plurality of linearly arranged transducer elements may be provided in the probe head 14.

Specifically, the ultrasound probe according to the embodiment is a so-called intraoperative probe. An object to be diagnosed is a liver, for example. During ultrasound diagnosis of the liver during operation, a wave transmitting and receiving surface of the probe head 14 is abutted against the exposed surface of the liver while the probe head 14 is held by a plurality of fingers of an operator. The probe head is held in abutment with the liver surface while it is manually scanned along the liver surface. In the course of the scanning, the scanning plane 18 is formed repeatedly, thereby obtaining a frame data array.

In the illustrated configuration example, the probe head 14 is provided with a magnetic sensor 20. A magnetic field (three-dimensional magnetic field) for positioning purpose is generated by a magnetic field generator 24, and this magnetic field is detected by the magnetic sensor 20. A detection signal output from the magnetic sensor 20 is transmitted to a positioning controller 26. The positioning controller 26 transmits a driving signal to the magnetic field generator 24. The positioning controller 26 calculates, based on the detection signal output from the magnetic sensor 20, a position and a posture of the probe head 14 in which the magnetic sensor 20 is provided. In other words, the positioning controller 26 calculates positional information of the scanning plane 18. In the embodiment, positional information is calculated for each received frame data set described below. The calculated positional information is output to a control unit 58.

The positioning controller 26 may be configured as an electronic circuit. The positioning controller 26 may be incorporated into the control unit 58. The magnetic sensor 20, the magnetic field generator 24, and the positioning controller 26 constitute a positioning system 28.

A transmission unit 30 is a transmission beam former that supplies, during transmission, a plurality of transmission signals in parallel, to the plurality of transducer elements constituting the array of transducer elements, and the transmission beam former is an electronic circuit. A reception unit 32 is a reception beam former that performs, during reception, phasing addition (delay addition) on a plurality of received signals output in parallel from the plurality of transducer elements constituting the array of transducer elements, and the reception beam former is an electronic circuit. The reception unit 32 is provided with a plurality of A/D converters, a detector circuit, and the like. The reception unit 32 performs phasing addition on the plurality of received signals, thereby generating beam data sets. Each received frame data set output from the reception unit 32 is composed of a plurality of beam data sets arranged in the electronic scanning direction. Each beam data set is composed of a plurality of echo data sets arranged in the depth direction. Although a beam data processing unit is provided downstream of the reception unit 32, it is omitted in the drawing.

A digital scan converter (DSC) 34 is an electronic circuit that forms a tomographic image based on the received frame data set. The DSC 34 has a coordinate conversion function, a pixel interpolation function, a frame rate conversion function, and the like. The DSC 34 transmits tomographic image data to an image processing unit 36, an identification unit 38, and a 3D memory 42. The tomographic image data are display frame data. The DSC 34 converts the received frame data array to a display frame data array.

The identification unit 38 performs identification processing on the tomographic image, on a frame-by-frame basis. A region of interest is set for the tomographic image. Within the tomographic image, an object that is subjected to the identification processing is an image portion defined by the region of interest. The identification processing is processing for automatically identifying, in the image portion, a tissue image that satisfies identification conditions. The identification result is transmitted to the image processing unit 36 and a tissue marker generation unit 40. The identification unit 38 is composed of an image processor, for example.

The tissue marker generation unit 40 generates, when a tissue image satisfying the identification conditions is identified, a tissue marker indicating the identified state and the identified tissue image. The tissue marker is a display element or a graphic figure. The tissue marker generation unit 40 transmits data of the tissue marker to the image processing unit 36. The tissue marker generation unit 40 is composed of an image processor, for example.

When the probe head 14 is manually scanned as described above, a plurality of tomographic image data sets (that is, a display frame data array) formed by manual scanning are stored in the 3D memory 42. They form a volume data set. Positional information obtained by the positioning system 28 is used when each display frame data set is written into the 3D memory 42.

A 3D memory 44 stores volume data sets obtained in the past from the same subject using other medical devices, as required. With the configuration according to the embodiment, it is possible to display a tomographic image of a certain cross section in real time while displaying another tomographic image of the same cross section in a parallel arrangement. A three-dimensional image may be displayed instead of the tomographic image. Other medical devices include an ultrasound diagnostic device, an x-ray CT scanner, an MRI scanner, and the like.

A region marker generation unit 46 generates a region marker indicating the region of interest. The region of interest is an elongated rectangular region that is set along the center line of the tomographic image. The region of interest is separated away from the upper edge and the lower edge of the tomographic image, and certain margins are provided above and below the region of interest. The image portion defined by the region of interest is also separated away from the upper edge and the lower edge of the tomographic image and has a rectangular shape elongated along the depth direction. Data of the region marker are transmitted to the image processing unit 36.

The image processing unit 36 functions as a display processing module. It is composed of an image processor, for example. The image processing unit 36 forms an image to be displayed on a display device 56. In addition to an image synthesizing function, the image processing unit 36 has a measurement function, an extraction function, a calibration function, an image forming function, and the like. In FIG. 1 these functions are respectively indicated as a measurement unit 48, an extraction unit 50, a calibration unit 52, and an image forming unit 54.

The measurement unit 48 performs, when a tissue image is identified, measurement on the tissue image. The concept of measurement encompasses size measurement, area measurement, and the like. The extraction unit 50 performs processing of extracting a three-dimensional tissue image from the volume data set using the result of identification of the tissue image. In the embodiment, a data set corresponding to the portal vein in the liver is extracted from the ultrasound volume data set. Another data set corresponding to the portal vein is already extracted as another volume data set. Two coordinate systems of the two volume data sets can be matched based on comparison between the extracted two data sets. This is performed by the calibration unit 52. The image forming unit 54 forms a tomographic image, a three-dimensional image, and the like based on each volume data set.

The display device 56 displays the tomographic image or the like as an ultrasound image. The display device 56 is composed of an LCD, an organic EL display device, or the like.

The control unit 58 controls operation of the individual elements shown in FIG. 1. The control unit 58 is composed of a CPU that executes a program. The CPU may realize a plurality of functions that are executed by the identification unit 38, the tissue marker generation unit 40, the image processing unit 36, the region marker generation unit 46, and the like. An operation panel 60 connected to the control unit 58 is an input device having a plurality of switches, a plurality of buttons, a track ball, a keyboard, or the like.

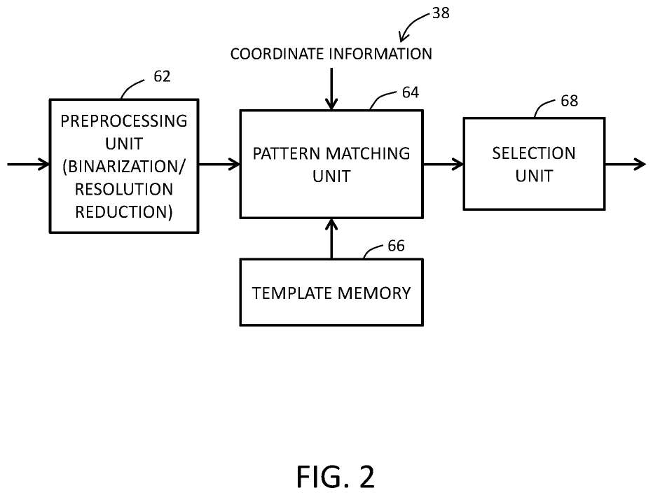

FIG. 2 shows a configuration example of the identification unit 38 shown in FIG. 1. The identification unit 38 identifies a tissue image satisfying the identification conditions by identification processing. Specifically, the identification unit 38 has a preprocessing unit 62, a pattern matching unit 64, a template memory 66, and a selection unit 68. The preprocessing unit 62 binarizes and reduces the resolution of a tomographic image to be processed (original image). In binarization, pixel values equal to or greater than a certain value are converted to 1, and pixel values smaller than the certain value are converted to 0. The resolution reduction performs thinning processing on the tomographic image to be processed, thereby reducing the tomographic image to, for example, ¼. It may be the case that the preprocessing is applied only to the region of interest or the image portion defined by the region of interest.

The preprocessed tomographic image is input to the pattern matching unit 64. The pattern matching unit 64 receives, as an input, coordinate information for identifying the coordinates of the region of interest. The template memory 66 stores templates used in the pattern matching processing. In the pattern matching processing, at least one type of template is used. Desirably, a plurality of types of templates are used simultaneously as described below.

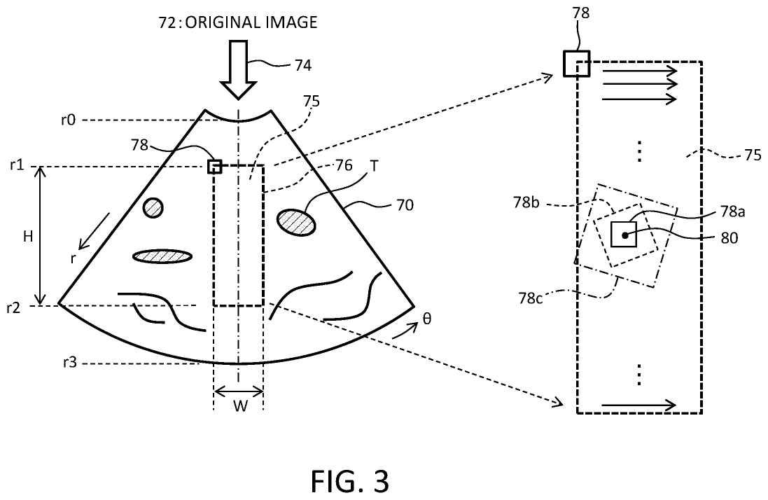

The pattern matching unit 64 performs the pattern matching processing at each of the positions within the region of interest. In the pattern matching processing, a correlation value (correlation coefficient) between the template and an object to be compared within the image portion is calculated. In practice, while sets of parameters, each set including a plurality of parameters (position, size, rotation angle, and the like), for the template are changed, a correlation value is calculated for each set of parameters. This will be described in detail with reference to FIG. 3.

The selection unit 68 identifies the best correlation value among a plurality of calculated correlation values and identifies a template; that is, a tissue image, corresponding to the best correlation value. As a correlation value, a Sum of Squared Difference (SSD), a Sum of Absolute Difference (SAD), or the like is known. The higher the degree of similarity between the two images, the closer the correlation value approaches 0. In the embodiment, a correlation value that is equal to or smaller than a threshold and closest to 0 is identified, and a tissue image is identified from this correlation value. It is also possible to use a correlation value that approaches 1 as the degree of similarity becomes higher. In either case, the pattern matching result is evaluated in terms of the degree of similarity.

Although, in the embodiment, one tissue image is identified in the identification processing, a plurality of tissue images may be identified simultaneously. That is, a plurality of tissue images satisfying the identification conditions may be identified in one image portion. In the embodiment, a tissue image that has generated the best correlation value equal to or smaller than the threshold is a tissue image satisfying the identification conditions. If no correlation value equal to or smaller than the threshold can be obtained, a determination is made that there is no tissue image satisfying the identification conditions. If a correlation value that approaches 1 as the degree of similarity becomes higher is used, a tissue image satisfying the identification conditions can be identified by identifying the largest correlation value that is equal to or larger than the threshold.

FIG. 3 schematically shows the pattern matching processing. A fan-shaped tomographic image 70 is shown on the left side of FIG. 3. The tomographic image 70 specifically shows a cross section of the liver. The tomographic image 70 includes a plurality of tissue images (a plurality of blood vessel cross section images). Among them, an image indicated by T is a target tissue image. The blood vessel cross section images other than that are other tissue images (non-target tissue images). The tomographic image 70 is an image generated by performing preprocessing 74 on an original image 72.

A region of interest 75 according to a first example is set on the tomographic image 70. An outer edge of the region of interest 75 is indicated by a region marker 76. The region of interest 75 defines a range or a portion to which the pattern matching processing is applied. More specifically, the region of interest 75 is an elongated rectangular region set on the central axis of the tomographic image 70 and is separated away from the upper edge and the lower edge of the tomographic image 70.

In FIG. 3, the lateral width of the region of interest 75 is indicated by W, and the vertical width (range of height) is indicated by H. The tomographic image 70 extends, on the central axis, from depth r0 to depth r3, and within this range, the region of interest 75 extends from depth r1 to depth r2. Although, in the embodiment, the display frame data after scan conversion are an object to be processed, the received frame data before scan conversion may be an object to be processed. Also, in that case, it is desirable to set, on the received frame data set, a region of interest having the shape shown in FIG. 3.

The enlarged region of interest 75 is shown on the right side in FIG. 3. The pattern matching processing is performed at positions within the region of interest 75. In other words, the pattern matching processing is performed sequentially while positions at which a template 78 is positioned are changed sequentially. These positions are positions at which the central coordinate of the template 78 is placed.

At each of the positions, a correlation value between the template and an object for comparison (image area on which the template is superimposed) is calculated while the size, the rotation angle, and the like of the template 78 are changed with the central coordinate of the template 79 fixed. In that case, only the size may be changed, both of the size and the rotation angle may be changed, or all of the size, the rotation angle, and the degree of deformation may be changed.

For example, at a position 80, the size and the rotation angle of the template are changed stepwise using the original template as a basis, thereby defining a plurality of derived templates 78a, 78b, and 78c, as shown. A correlation value is calculated for each individual derived template. Such template processing is performed over the entire region of interest 75.

Finally, the best correlation value equal to or smaller than the threshold is identified, based on which a tissue image is identified. Tissue image identification is performed on a frame-by-frame basis; that is, new identification processing is performed when the frames are switched. For a frame having no correlation value equal to or smaller than the threshold (that is, no similarity above a certain level), tissue image identification is not carried out.

In the embodiment, within the tomographic image 70, an area to be compared with the template 78 is, in the strict sense, an image portion that is larger than the region of interest 75. In other words, the image portion is a portion that is referred to in pattern matching. The image portion is of a size larger than the region of interest 75. Of course, it is also possible to conduct a search for a tissue image only within the region of interest 75. In that case, the image portion and the region of interest 75 match. The image portion is usually separated away from the upper edge and the lower edge of the tomographic image 70.

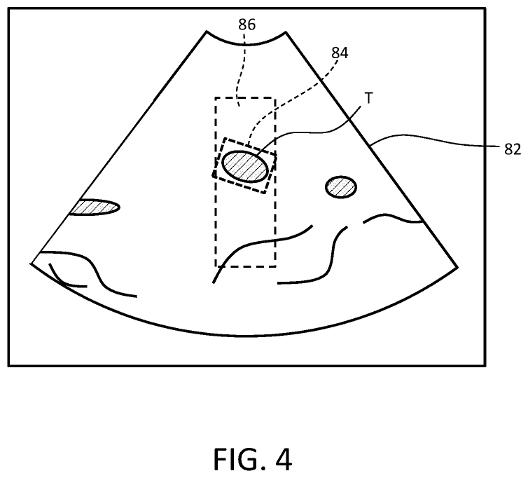

FIG. 4 shows an identified target tissue image T included in a tomographic image 82. In the illustrated example, the target tissue image T is included in a region of interest 86. A rectangular tissue marker 84 is displayed so as to enclose the target tissue image T. It indicates an outer edge of a template used when the best matching state is obtained. Through observation of the tissue marker 84, it becomes possible for the examiner to recognize the identified state and the identification target. When the identified state is achieved, display of the region marker indicating the outer edge of the region of interest 86 may be stopped.

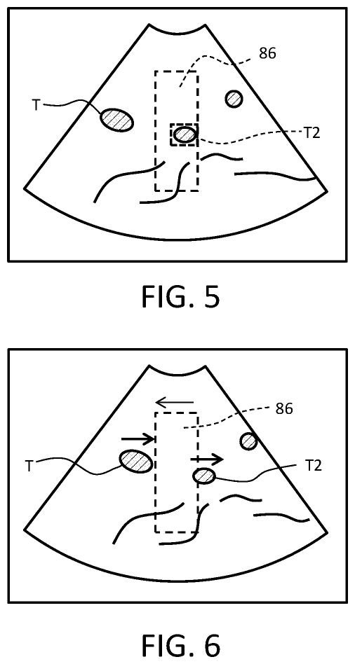

FIG. 5 shows another identified tissue image T2 which is not the target tissue image T. The tissue image T2 is within a region of interest 86, and the target tissue image is outside of the region of interest 86. In such a case, the probe may be translated on the body surface as shown in FIG. 6. That is, the scanning plane may be translated while the orientation of the scanning plane is maintained. When the tissue image T2 is outside of the region of interest 86, the tissue image T2 is no longer an identification target or an identification candidate. If the target tissue image T enters the region of interest 86, it becomes a new identification target.

For example, after a target blood vessel is identified as a target tissue image on a tomographic image, the probe head may be translated along the target blood vessel. Such manual scanning allows the target blood vessel to be extracted as a plurality of target tissue images. Alternatively, after a target blood vessel is identified as a target tissue image on a tomographic image, and the user makes a predetermined input, a three-dimensional target blood vessel image may be extracted from a volume data set using the input as a trigger.

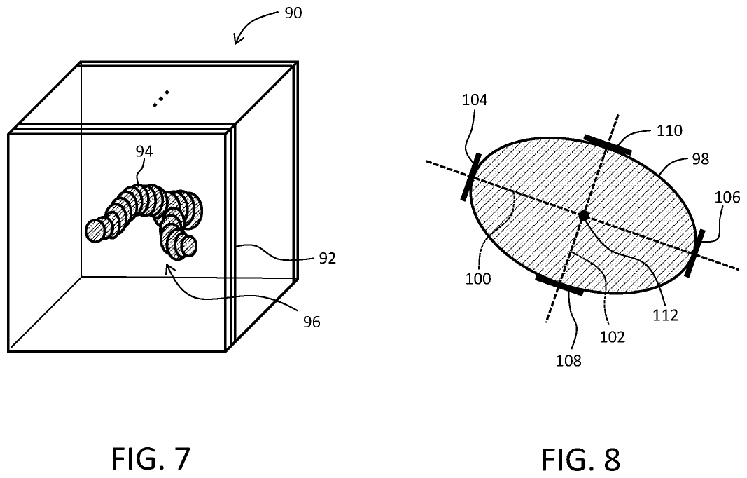

FIG. 7 shows an example of processing subsequent to the identification processing (subsequent processing). A volume data set 90 is composed of a plurality of display frame data sets 92. When a target tissue image 94 is automatically identified on a particular display frame data set selected from the plurality of display frame data sets 92, a target tissue image may be identified on each frame data set using a connection relationship having the identified target tissue image 94 as a starting point. Finally, a three-dimensional target tissue image 96 is extracted.

FIG. 8 shows another example of the subsequent processing. Two axes 100 and 102 are automatically set for a target tissue image 98 using a set of parameters used when a template fits the target tissue image 98. The size of the target tissue image 98 is measured on each of the axes 100 and 102 using an edge detection technique or the like. At that time, the area and the like may also be calculated.

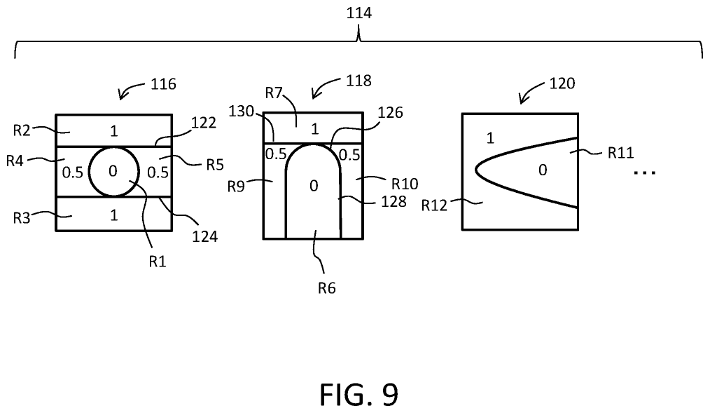

FIG. 9 shows an example of a set of templates. On a tomographic image, a target tissue image may appear in various appearances, and therefore, a set of templates that includes a plurality of templates is used. A set of templates 114 shown in FIG. 9 includes a first template 116, a second template 118, and a third template 120. They are used to identify a particular blood vessel image.

The first template 116 has a rectangular shape as a whole and includes a circular region R1 that simulates a cross section of a blood vessel. Above and below the region R1, there are horizontally elongated regions R2 and R3 that are in contact with the region R1. There are regions R4 and R5 outside the region R1 and sandwiched between the regions R2 and R3. The region R1 has a value of 0, and the regions R2 and R3 have a value of 1. The regions R4 and R5 have a value of 0.5. The regions R4 and R5 are treated as neutral regions in terms of calculation of correlation values. This takes into consideration that an oblique cross section (cross section extending in the lateral direction) of the blood vessel may appear. Reference numerals 122 and 124 indicate parting lines between the regions.

The second template 118 has a rectangular shape as a whole and includes a region R6 therein. The region R6 has a shape in which a circle 126 corresponding to the blood vessel is connected to a shadow 128 generated on the lower side of the circle 126. A circular blood vessel image tends to have a shadow generated on the lower side thereof, and therefore, this shape is for extracting such a blood vessel image with a shadow. Because a region of interest is set on the center portion of a tomographic image, within the region of interest, a shadow is generated generally directly below an object. The shadow is a portion at which the echo intensity is weak and is a portion displayed in black on the tomographic image. The second template 118 does not have to be rotated.

There are a region R7 on the upper side of the region R6 and regions R9 and R10 on the respective sides of the region R6 and below the region R7. The region R6 has a value of 0, and the region R7 has a value of 1. The regions R9 and R10 have a value of 0.5. This takes into consideration that an oblique cross section of the blood vessel with a shadow may appear.

The third template 120 simulates an oblique cross section of the blood vessel and includes two regions R11 and R12. The region R11 has a value of 0, and the region R12 has a value of 1.

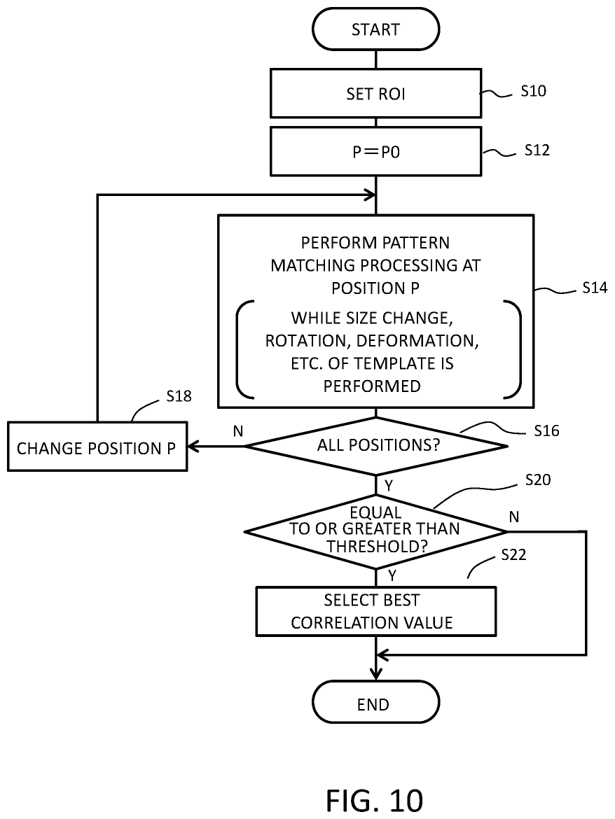

FIG. 10 shows a flowchart of the identification processing according to the embodiment. The identification processing is performed on a frame-by-frame basis.

In S10, a region of interest (ROI) is set on a tomographic image. In S12, a position P within the region of interest is initialized In S14, the pattern matching processing is performed at the position P. The pattern matching processing is performed so as to execute pattern matching a plurality of times (correlation is calculated a plurality of times) while changing the size and the rotation angle of the template and deforming the template. If a plurality of templates are used, the pattern matching processing is performed for each template.

In S16, a determination is made as to whether or not pattern the matching processing has been performed for all the positions in the region of interest, and if the processing has not been completed yet, the position P is changed in S18, and then, the processing in S14 is performed again. In S20, a determination is made as to whether or not, among the plurality of calculated correlation values, there is any correlation value that is equal to or smaller than a threshold (good correlation value). If there is at least one such a correlation value, in S22, the smallest correlation value is identified, and a tissue image satisfying the identification conditions is identified based on a set of parameters corresponding to that correlation value. The above identification processing is performed for each frame.

The examiner adjusts a position and a posture of the probe head so that the target tissue image is included in the region of interest, and a non-target tissue image, for which erroneous identification is likely to be made, is excluded from the region of interest. As a result, the target tissue image can be easily identified automatically.

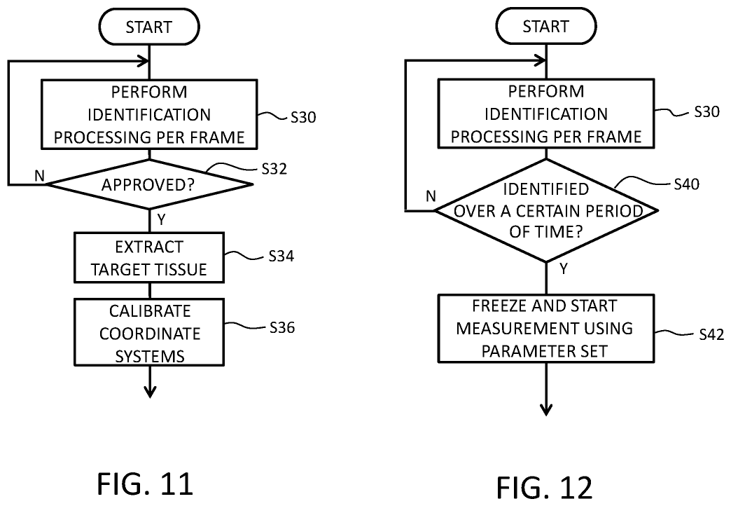

FIG. 11 shows a first example of processing subsequent to the identification processing. In S30, the identification processing is performed on a frame-by-frame basis. If, in S32, a user operation for approving the identified tissue image is applied, in S34, a three-dimensional tissue image is extracted from a volume data set using the identified tissue image as a starting point. In S36, calibration is performed to match the coordinate systems between two volume data sets, based on the extracted three-dimensional tissue image.

FIG. 12 shows a second example of processing subsequent to the identification processing. S30 is the same as that shown in FIG. 11, and the description thereof will be omitted. In S40, a determination is made as to whether the same tissue image has been continuously identified over a certain period of time. In S42, the tomographic image is frozen, and measurement of the tissue image is automatically performed using the set of parameters. According to this second example, a series of processing steps from identification to measurement of the target tissue image is performed automatically, and therefore, the burden imposed on the user is reduced significantly.

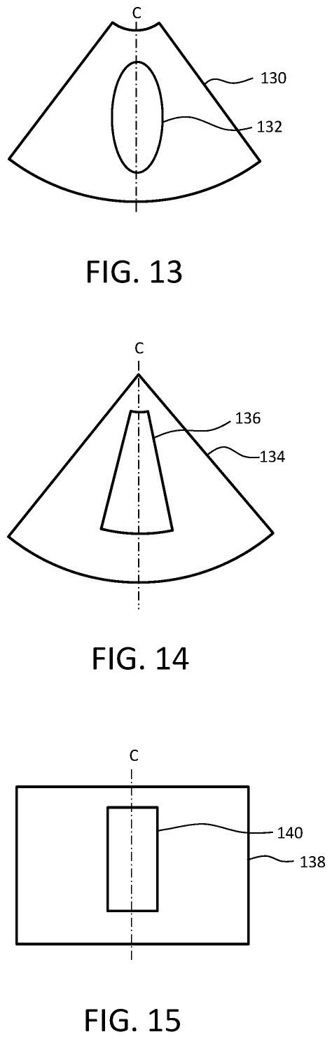

FIG. 13 shows a second example of the region of interest. An elongated oval region of interest 132 is set on the center line C of a fan-shaped tomographic image 130. Specifically, the major axis of the region of interest 132 matches the center line C, and the minor axis thereof is orthogonal to the center line C.

FIG. 14 shows a third example of the region of interest. An elongated fan-shaped region of interest 136 is set on the center line C of a fan-shaped tomographic image 134. The region of interest 136 is defined according to the polar coordinate system, for example.

FIG. 15 shows a fourth example of the region of interest. An elongated rectangular region of interest 140 is set on the center line C of a rectangular tomographic image 138.

As described above, according to the embodiment, an elongated region of interest extending in the depth direction is set in the center of a tomographic image. If a tissue image satisfying target conditions is included in the region of interest (in the strict sense, in an image portion), that tissue image is identified automatically. Such identification can be easily achieved by adjusting a position and a posture of the probe head, and therefore, no significant burden is imposed on the examiner. If the identified tissue image is erroneous; that is, if the tissue image is not the target tissue image, the position and the posture of the probe head only need to be changed so that that tissue image is outside of the image portion. Thus, the tissue image is excluded from identification targets in the course of nature. As such, according to the embodiment, it is possible to select an identification target easily by operating the probe head.

1. An ultrasound diagnostic device comprising:
a probe head that transmits and receives ultrasound waves;
an image forming unit that forms an ultrasound image based on a received signal output from the probe head;
a region setting unit that defines a region of interest extending in the depth direction with respect to the ultrasound image;
an identification unit that identifies, in an image portion defined by the region of interest, a tissue image that satisfies identification conditions; and
a tissue marker generation unit that generates, when the tissue image satisfying the identification conditions is identified, a tissue marker indicating the tissue image and causes the tissue marker to be displayed on the ultrasound image,
wherein when the tissue image that has been identified so far is outside of the image portion in accordance with operation of the probe head, the tissue image is excluded from identification targets.

2. The ultrasound diagnostic device according to claim 1, wherein the region of interest is provided on a center line of the ultrasound image and has an elongated shape extending along the center line.

3. The ultrasound diagnostic device according to claim 2, wherein
the ultrasound image has a fan shape, and
the region of interest has a rectangular shape separated away from an upper edge and a lower edge of the ultrasound image.

4. The ultrasound diagnostic device according to claim 1, wherein
the identification unit repeats identification processing on a frame-by-frame basis, and
in the identification processing on a frame-by-frame basis, pattern matching processing using at least one template is performed at positions within the region of interest, and a tissue image satisfying the identification conditions is identified based on a plurality of pattern matching results obtained from the pattern matching processing.

5. The ultrasound diagnostic device according to claim 4, wherein in the pattern matching processing, a set of templates that comprises a plurality of templates different from one another is used.

6. The ultrasound diagnostic device according to claim 5, wherein the set of templates includes a template that simulates a tissue image with a shadow.

7. The ultrasound diagnostic device according to claim 4, wherein the pattern matching processing at the positions in the region of interest includes at least one of change in template size, change in template rotation angle, and template deformation.

8. The ultrasound diagnostic device according to claim 1, further comprising a region marker generation unit that generates a region marker indicating the region of interest and causes the region marker to be displayed on the ultrasound image.

9. An ultrasound image processing method comprising the steps of:
setting, with respect to an ultrasound image, a region of interest extending on a center line of the ultrasound image in the depth direction, the ultrasound image being formed based on a received signal output from a probe head transmitting and receiving ultrasound waves;
identifying, in an image portion defined by the region of interest, a tissue image that satisfies identification conditions;
displaying a region marker indicating the region of interest on the ultrasound image; and
displaying a tissue marker indicating, on the ultrasound image, an identified state of the tissue image satisfying the identification conditions.
